(19) United States
(12) Patent Application Publication
(10) Pub. No.: US 2009/0098069 A1
(43) Pub. Date: Apr. 16, 2009

(54) TRANSDERMAL, ALCOHOL-FREE, PHARMACEUTICAL COMPOSITIONS

(75) Inventor: Rita Downard Vacca, Frontenac, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 12/209,864

(22) Filed: Sep. 12, 2008

Related U.S. Application Data
(60) Provisional application No. 60/993,874, filed on Sep. 14, 2007.

(51) Int. Cl. A61K 8/30; A61P 17/00; A61K 31/56; A61K 31/565; A61K 31/568

(52) U.S. Cl. 424/59; 514/772; 514/178; 514/182; 514/784; 514/785

(57) ABSTRACT

An alcohol-free, transdermal drug delivery composition administered via a metered spray drug delivery device is described herein. The non-occlusive transdermal drug delivery composition includes a therapeutically effective amount of at least one physiologically active agent or prodrug thereof, an effective amount of at least one dermal penetration enhancer; and at least one non-volatile liquid. The transdermal drug delivery composition is administered to a dermal or mucosal surface of an animal needing the same using a metered spray device capable of delivering a fine spray of substantially uniform particle size to minimize the required drying time therefor.

This application claims the benefit of U.S. provisional patent application Ser. No. 60/993,874, filed on Sep. 14, 2007, the entire disclosure of which is incorporated by reference herein.

### FIELD OF THE INVENTION

The present invention relates to alcohol-free pharmaceutical compositions, and more particularly to alcohol-free, non-occlusive, transdermal pharmaceutical compositions preferably administered to a dermal surface via a metered spray device.

### BACKGROUND OF THE INVENTION

Dermal delivery of drugs may represent the oldest form of drug delivery in human history. Resins and animal fats were probably used by humans in early times to treat damage to the skin resulting from injuries and burns. Such substances for local delivery of active substances remained largely unchanged until as late as this century.

The prevention or treatment of local or topical disease states or conditions of the skin has traditionally used simple non-occlusive delivery systems. These drug delivery systems usually include a volatile and/or non-volatile medium whereby a composition of the drug and medium is topically applied to the skin, generally in the vicinity of or directly on the area of skin to be treated. Such delivery systems usually take the form of emulsions, creams, ointments, foams, gels, liquids, sprays and aerosols. These delivery systems are generally used to treat skin inflammations, soft-tissue contusions, parasites, fungal and bacterial topical infection and topical analgesia. The limitation with this type of delivery system is that systemic drugs are generally not suitable for this type of administration.

Some major problems with the current state of the art are based on the lack of efficacy of transdermally delivered systemic drugs. Systemic drugs lack efficacy transdermally due to the low drug flux across the skin, as observed for drugs such as testosterone, amlodipine, fentanyl, buprenorphine and many other drugs. Other drugs, such as glyceryl trinitrate, Nitrobid™ (a drug for the treatment of angina), are difficult to deliver by transdermal systems due to the inability to adequately control the rate of drug delivery, or the requirement for a very large application area. Other problems with the poor dermal penetration of drugs is that the drug can be easily washed off or transferred to clothes, other surfaces or other animals.

The concept of transdermal systemic drug delivery was first seriously advocated in the 1970's by Dr. Alejandro Zaffaroni in U.S. Pat. Nos. 3,598,122; 3,731,683 and 3,797,494. Transdermal systemic drug delivery provides an effective method of achieving improved bioavailability for physiologically active substances where the drugs are poorly absorbed by traditional routes of delivery. It can also be used where oral dosing is poorly tolerated or not possible.

Transdermal formulations are however limited. For example, polar drugs tend to penetrate the skin too slowly. Since most drugs are of a polar nature, this limitation is significant. Another significant factor is that many drugs can cause irritation at the site of topical application.

Two main methods are known for assisting the rate of penetration of drugs across the skin. The first method is to increase the thermodynamic activity of the drug. The thermodynamic activity of a drug in a dermal formulation is proportional to the concentration of the drug and the selection of the vehicle. According to the laws of thermodynamics, the maximum activity of a drug is related to that of the pure drug crystal. The second method involves the use of compounds known as penetration enhancers to increase the permeability of the dermal surface and has generally proven to be more convenient and effective.

Since the early 1970s, the main focus of transdermal systemic drug delivery has been, and still is, on transdermal patch devices. These patch devices are like bandages which are attached to the surface of intact skin for prolonged periods of time to allow a desired systemic delivery of a drug or other physiologically active agent. These transdermal patch devices occlude the skin and trap the drug, together with volatiles and vehicle excipients, between the skin and an outer impermeable backing membrane. The backing membrane prevents the evaporation or diffusion of vehicle excipients, volatiles and drug into an environment other than the target skin site. The prolonged length of time required for transfer of the drug and excipients from the patch into the skin can and often does result in local skin irritation. The irritation is caused by prolonged contact on the skin by the drug, volatiles, vehicle excipients, and/or the adhesive used to attach the patch device to the skin. The occlusive nature of the patch device also restricts the natural ability of the skin to “breathe”, increasing the risk of irritation. With added problems of complex and costly manufacturing processes for transdermal patch devices there, is a need for improved transdermal drug delivery systems.

One method of improving transdermal drug delivery systems is to increase the rate of drug delivery across a dermal surface. Drug delivery across a dermal surface can be increased by dermal penetration enhancers. The problem with most known dermal penetration enhancers is that they are often toxic, irritating or allergenic. Dermal penetration enhancers tend to be proton accepting solvents such as dimethylsulfoxide and dimethyacetamide. More recently, 2-pyrrolidine, N,N-diethyl-m-toluamide (Deet®), 1-dodecal-azacycloheptane-2-one (Azone®), N,N-dimethylformamide, N-methyl-2-pyrrolidine and calcium thioglycolate have been reported as effective dermal penetration enhancers. However, difficulties remain with such dermal penetration enhancers because the problem of irritation at the site of application has not been overcome.

The most critical problem with dermal penetration enhancers however is toxicity. If a compound when used as a dermal penetration enhancer is toxic, irritating or allergenic, then that compound is unsuitable for application to the animal body. Dimethyl sulfoxide and dimethyl acetamide are not clinically acceptable for these reasons. Although Deet® and Azone® have reportedly lower toxicities, their toxicity is still such that they are not widely used. It is possible that Azone® may be employed as a dermal penetration enhancer if the amount applied is sufficiently small so as not to be appreciably toxic, irritating or allergenic to the animal.

The thermodynamic activity of a drug can be increased by employing supersaturated systems which give rise to unusually high thermodynamic potentials [Coldman, et al., J. Pharm. Sci., 58(9), 119, 1969]. However, topical vehicles relying on supersaturation, have the major limitation of formulation instability, both prior to and during application to the skin. As such, they are of limited clinical value within a non-occlusive volatile:non-volatile delivery vehicle, because as soon as the formulation comes into contact with a person's clothing or the like, the drug often precipitates; hence the formulation is no longer supersaturated and any enhanced percutaneous absorption ceases.

Others, such as Kondo, et al., [J. Pharmacobio-Dyn., 10, 743, 1987], using supersaturation to achieve enhanced transdermal drug delivery, relied on the use of anti-nucleating polymers to stabilize the formulation. However, the applied drug formulations stabilized with polymers formed an appreciable surface mass on the skin which remained there over a prolonged period of many hours, not a few minutes. So while Kondo advocated the use of a metered spray to deliver these formulations, in reality it would be impossible to obtain a non-occlusive delivery system with a short application time and still maintain a clinically useful transdermal pharmaceutical product.

German patent application DE 433-4553-A1 assigned to Jenapharm GmbH discloses a pharmaceutical liquid system consisting of a drug (diclofenac), a lipophilic phase, a volatile component and appropriate antioxidants, preservatives or stabilizers. This system relies on supersaturation to increase the flux rate of dermal absorption. An application chamber is used to prevent accidental precipitation of the supersaturated drug delivery system over the application time of 150 minutes.

Japanese patent JP 61-268631 assigned to Showa Denko KK discloses dermal penetration enhancers suitable for use with water-soluble drugs. The dermal penetration enhancers disclosed include 1-5 carbon fatty acid esters of para-aminobenzoic acid. The preferred dermal penetration enhancer disclosed in JP 61-268631 is the 2 carbon fatty acid ester of para-aminobenzoic acid (Benzocaine®). The preferred dermal penetration enhancer disclosed in JP 61-268631 has significant pharmacological properties in that it is a local anaesthetic, which has also been reported to cause skin irritation and allergic skin reactions.

It is not surprising that previous studies, e.g., [Feldmann, et al., Arch. Derm., 94, 649, 1996; Coldman, et al., J. Pharm. Sci., 58(9), 119, 1969; and Bhatt, et al., Int. J. Pharm., 50, 157, 1989], where low volumes of non-occlusive, volatile:non-volatile vehicles were applied to the skin, the level of drug delivered was very limited. To date, clinically useful formulations tend to be local therapies, such as topical minoxidil, topical non-steroidal anti-inflammatories, or transdermally delivered drug compounds which readily diffuse across the skin such as glyceryl trinitrate and isosorbide dinitrate. As the permeability coefficient of sex hormones, for example, are an order of magnitude lower than glyceryl trinitrate, a marked penetration enhancement effect would be needed to achieve clinically acceptable transdermal drug delivery.

It is desirable to have a clinically acceptable non-occlusive, transdermal drug delivery system where the drug and penetration enhancer undergo rapid partitioning into the skin to allow a convenient application time, leaving no residual formulation on the skin surface, and maintaining good substantivity within the skin. These characteristics can overcome problems such as a loss of drug penetration or possibly a transfer of the drug from the treated individual to another upon intimate contact, such as that observed for a testosterone ointment being used for a male patient, but which caused virilization in his female sexual partner [Delance, et al., Lancet, 1, 276, 1984].

It is also desirable to have a clinically acceptable non-occlusive, transdermal drug delivery system that is alcohol-free. Manufacturing products containing volatile liquids can be a safety concern during manufacture. Avoiding the use of volatile liquids may also be more economical to manufacture due to the reduction or elimination of many safety related issues.

It is an object of the present invention to overcome or at least alleviate one or more of the above-mentioned disadvantages of the prior art systems.

### SUMMARY OF THE INVENTION

According to a first aspect of the present invention there is provided an alcohol-free, non-occlusive, transdermal drug delivery composition comprising an alcohol-free composition with a therapeutically effective amount of at least one physiologically active agent or prodrug thereof and an effective amount of at least one dermal penetration enhancer. The drug delivery composition of the present invention is preferably administered to a dermal surface in need thereof via a metered dose drug delivery system having a pressure actuated-valve of predetermined dimensions. Most importantly, the vapor tap and stem orifice sizes of the pressure actuated-valve are of predetermined dimensions to produce a fine, relatively “dry” spray of the composition to an intended dermal surface in need thereof. Aerosol administration of the subject drug delivery composition includes packaging in a suitable aerosol device a composition of the present invention with the following ingredients:
(a) an effective amount of at least one physiologically active agent or prodrug thereof;
(b) an effective amount of one or more permeation enhancers;
(c) one or more suitable propellants in an amount in excess of 35 weight % of total device fill; and
(d) one or more non-volatile liquids in the amount of the remainder of the total device fill

In accordance with the present invention, the excess propellant, most preferably dimethyl ether, enables the delivery of a fine, soft spray at a predetermined substantially constant spray rate which is of a substantially uniform particle size and composition, without clogging, during delivery of each metered dose throughout substantially the entire fill contents of the aerosol device and at substantially a constant pressure. Such is most preferably achieved using predetermined device dimensions as set forth below.

Actuator-Valve Dimensions

Vapor tap: about 0.013 inches to about 0.020 inches

Stem orifice: about 0.010 inches to about 0.014 inches

Nozzle orifice: about 0.018 inches±10% inches

The defined composition administered to a dermal surface using a drug delivery device with the particular vapor tap, nozzle and stem orifice dimensions noted above, provides an advantageous spray delivery rate of about 0.20-0.25 g/second. The particle size of the spray is about 50 microns+10 microns. Such produces a relatively “dry” spray mist dosage requiring approximately four minutes or less to dry on a dermal surface under ambient conditions and humidity.

### BRIEF DESCRIPTION OF THE DRAWINGS

FIG. 1 is a side view of a drug delivery device of the present invention, have the following components: can (10), contents (12), vapor tap (14), valve actuator (16), valve (18), stem orifice (20), capillary tubing (22), and exit orifice (24).

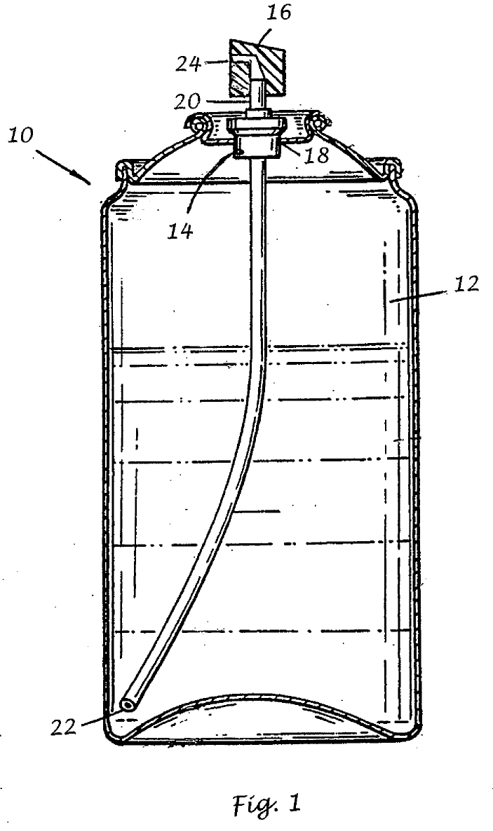

### DETAILED DESCRIPTION OF THE INVENTION

In accordance with the present invention, alcohol-free, non-occlusive, pharmaceutical compositions are described. Compositions of the present invention comprise (i) a therapeutically effective amount of at least one physiologically active agent or prodrug thereof, (ii) an effective amount of at least one dermal penetration enhancer; and (iii) at least one non-volatile liquid; characterised in that the dermal penetration enhancer is adapted to transport the physiologically active agent across a dermal surface or mucosal membrane of an animal, including a human, when the non-volatile liquid evaporates in about four minutes or less, to form a reservoir or depot of a mixture comprising the penetration enhancer and the physiologically active agent or prodrug within said surface or membrane. The preferred dermal penetration enhancer of the present invention is of low toxicity so as to be well tolerated by the dermal surface or mucosal membrane of the animal.

Suitable physiologically active agents or prodrugs thereof include those that are soluble in the non-volatile liquid portion of the composition of the present invention. Physiologically active agents or prodrugs thereof that are not readily soluble in the non-volatile liquid portion of the composition may be modified to increase the solubility thereof by one of many approaches known to those skilled in the art, such as for example but not limited to, transforming a crystalline active agent to its amorphous form and then stabilizing the amorphous form. Another method by which solubility of an active agent or prodrug may be increased is by micronizing or nanosizing the active agent or prodrug particles thereby significantly increasing the surface area thereof and increasing solubility. Suitable modified or unmodified physiologically active agents or prodrugs thereof include physiologically active agents that may be used in the preferred transdermal, or alternatively, a percutaneous drug delivery system of the present invention including any locally or systemically active agents which can be delivered through the skin with the assistance of one or more dermal penetration enhancers to achieve a desired effect. The physiologically active agents may be selected from androgens, estrogens, or progestogens or any combination thereof, for example, androgens plus estrogens, androgens plus progestogens, or androgens plus estrogens, plus progestogens, provided that when the active agent is an estrogen or a progestogen, a therapeutically effective amount of a progestogen or estrogen, respectively, is not present in the formulation. Particularly preferred active agents include: androgens, anti-androgens, estrogens, anti-estrogens, progestogens, anti-progestogens, adrenergic agonists, analgesics, sedatives, amides, arylpiperazines, nerve agents, antineoplastics, anti-inflammatory agents, anticholinergics, anticonvulsants, antidepressants, antiepileptics, antihistaminics, antihypertensives, muscle relaxants, diuretics, bronchodilators, and glucocorticoids. If desired, the active agent may be present in combination with a secondary active agent for concurrent administration subject to the previously stated provision. Additional examples of such physiologically active agents not intended to be limiting (grouped by therapeutic class) include:

### Alimentary System:

Antidiarrhoeals such as diphenoxylate, loperamide and hyoscyamine.

### Cardiovascular System:

Antihypertensives such as hydralazine, minoxidil, captopril, enalapril, clonidine, prazosin, debrisoquine, diazoxide, guanethidne, methyldopa, reserpine, trimetaphan.

Calcium channel blockers such as diltiazem, felodopine, amlodipine, nitrendipine, nifedipine and verapamil.

Antiarrhyrthmics such as amiodarone, flecamide, disopyramide, procainamide, mexiletene and quinidine.

Antiangina agents such as glyceryl trinitrate, erythritol tetranitrate, pentaerythritol tetranitrate, mannitol hexanitrate, perhexylene, isosorbide dinitrate and nicorandil.

Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate.

Cardiotonic glycosides such as digoxin and other cardiac glycosides and theophylline derivatives.

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine.

Vasodilators such as cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate and xanthinol.

Antimigraine preparations such as ergotamine, dihydroergotamine, methysergide, pizotifen and sumatriptan.

### Drugs Affecting Blood and Haemopoietic Tissues:

Anticoagulants and thrombolytic agents such as warfarin and dicoumarol.

Low molecular weight heparins such as enoxaparin, streptokinase and its active derivatives.

Haemostatic agents such as aprotinin, tranexamic acid and protamine.

### Central Nervous System;

Analgesics, antipyretics including the opiod analgesics such as buprenorphine, dextromoramide, dextropropoxyphene, fentanyl, alfentanil, sufentanil, hydromorphone, methadone, morphine, oxycodone, papavereturn, pentazocine, pethidine, phenoperidine, codeine and dihydrocodeine. Others include acetylsalicylic acid (aspirin), paracetamol, and phenazone.

Hypnotics and sedatives such as the barbiturates, amylobarbitone, butobarbitone and pentobarbitone and other hypnotics and sedatives such as choral hydrate, chlormethiazole, hydroxyzine and meprobamate.

Antianxiety agents such as the benzodiazepines, alprazolam, bromazepam, chlordiazepoxide, clobazam, chlorazepate, diazepam, flunitrazepam, flurazepam, lorazepam, nitrazepam, oxazepam, temazepam and triazolam.

Neuroleptic and antipsychotic drugs such as the phenothiazines, chlorpromazine, fluphenazine, pericyazine, perphenazine, promazine, thiopropazate, thioridazine and trifluoperazine and the butyrophenones, droperidol and haloperidol and the other antipsychotic drugs such as pimozide, thiothixene and lithium.

Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline.

CNS stimulants such as caffeine.

Anti-alzheimer's agents such as tacrine.

Antiparkinson agents such as amantadine, benserazide, carbidopa, levodopa, benztropine, biperiden, benzhexyl, procyclidine and dopamine-2 agonists such as S(−)-2-(N-propyl-N-2-thienylethylamino)-5-hydroxytetralin (N-0).

Anticonvulsants such as phenyloin, valproic acid, primidone, phenobarbitone, methylphenobarbitone and carbamazepine, ethosuximide, methsuximide, phensuximide, sulthiame and clonazepam.

Antiemetics, antinauseants such as the phenothiazines, prochloperazine, thiethylperazine and 5HT-3 receptor antagonists such as ondansetron and granisetron and others such as dimenhydrinate, diphenhydramine, metoclopramide, domperidone, hyoscine, hyoscine hydrobromide, hyoscine hydrochloride, clebopride and brompride.

### Musculoskeletal System:

Non-steroidal anti-inflammatory agents including their racemic mixtures or individual enantiomers where applicable, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol and ketoralac.

Additional non-steroidal antiinflammatory agents which can be formulated in combination with the dermal penetration enhancers include salicylamide, salicylic acid, flufenisal, salsalate, triethanolamine salicylate, aminopyrine, antipyrine, oxyphenbutazone, apazone, cintazone, flufenamic acid, clonixeril, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloide, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

Antirheumatoid agents such as penicillamine, aurothioglucose, sodium aurothiomalate, methotrexate and auranofin.

Muscle relaxants such as baclofen, diazepam, cyclobenzaprine hydrochloride, dantrolene, methocarbamol, orphenadrine and quinine.

Agents used in gout and hyperuricaemia such as allopurinol, colchicine, probenecid and sulphinpyrazone.

### Hormones and Steroids:

Oestrogens such as oestradiol, oestriol, oestrone, ethinyloestradiol, mestranol, stilboestrol, dienoestrol, epioestriol, estropipate and zeranol.

Progesterone and other progestagens such as allyloestrenol, dydrgesterone, lynoestrenol, norgestrel, norethyndrel, norethisterone, norethisterone acetate, gestodene, levonorgestrel, medroxyprogesterone and megestrol.

Antiandrogens such as cyproterone acetate and danazol.

Antioestrogens such as tamoxifen and epitiostanol and the aromatase inhibitors, exemestane and 4-hydroxy-androstenedione and its derivatives. Androgens and anabolic agents such as testosterone, methyltestosterone, clostebol acetate, drostanolone, furazabol, nandrolone oxandrolone, stanozolol, trenbolone acetate, dihydro-testosterone, 17-.alpha.-methyl-19-nortestosterone and fluoxymesterone.

5-alpha reductase inhibitors such as finasteride and turosteride.

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate methylprednisolone, prednisolone, prednisolone 21-phosphate, prednisone, triamcinolone, triamcinolone acetonide.

Further examples of steroidal antiinflammatory agents for use in the instant compositions include include cortodoxone, fluoracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

Pituitary hormones and their active derivatives or analogs such as corticotrophin, thyrotropin, follicle stimulating hormone (FSH), luteinising hormone (LH) and gonadotrophin releasing hormone (GnRH).

Hypoglycaemic agents such as insulin, chlorpropamide, glibenclamide, gliclazide, glipizide, tolazamide, tolbutamide and metformin.

Thyroid hormones such as calcitonin, thyroxine and liothyronine and antithyroid agents such as carbimazole and propylthiouracil.

Other miscellaneous hormone agents such as octreotide.

Pituitary inhibitors such as bromocriptine.

Ovulation inducers such as clomiphene.

### Genitourinary System:

Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potassium sparing diuretics, spironolactone, amiloride and triamterene.

Antidiuretics such as desmopressin, lypressin and vasopressin including their active derivatives or analogs.

Obstetric drugs including agents acting on the uterus such as ergometrine, oxytocin and gemeprost.

Prostaglandins such as aiprostadil (PGE1), prostacyclin (PGI2), dinoprost (prostaglandin F2-alpha) and misoprostol.

### Antimicrobials:

Antimicrobials including the cephalosporins such as cephalexin, cefoxytin and cephalothin.

Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, mezlocillin, piperacillin, ticarcillin and azlocillin.

Tetracyclines such as minocycline, chlortetracycline, tetracycline, demeclocycline, doxycycline, methacycline and oxytetracycline and other tetracycline-type antibiotics.

Aminoglycosides such as amikacin, gentamicin, kanamycin, neomycin, netilmicin and tobramycin. Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione.

Quinolones such as nalidixic acid, cinoxacin, ciprofloxacin, enoxacin and norfloxacin. Sulphonamides such as phthalylsulphthiazole, sulfadoxine, sulphadiazine, sulphamethizole and sulphamethoxazole.

Sulphones such as dapsone.

Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonam, colistin IV, metronidazole, timidazole, fusidic acid and trimethoprim, 2-thiopyridine N-oxide; halogen compounds, particularly iodine and iodine compounds such as iodine-PVP complex and diiodohydroxyquin; hexachlorophene; chlorhexidine; chloroamine compounds; benzoylperoxide.

Antituberculosis drugs such as ethambutol, isoniazid, pyrazinamide, rifampicin and clofazimine. Antimalarials such as primaquine, pyrimethamine, chloroquine, hydroxychloroquine, quinine, mefloquine and halofantrine.

Antiviral agents such as acyclovir and acyclovir prodrugs, famciclovir, zidovudine, didanosine, stavudine, lamivudine, zalcitabine, saquinavir, indinavir, ritonavir, n-docosanol, tromantadine and idoxuridine.

Anthelmintics such as mebendazole, thiabendazole, niclosamide, praziquantel, pyrantel embonate and diethylcarbamazine.

Cytotoxic agents such as plicamycin, cyclophosphamide, dacarbazine, fluorouracil and its prodrugs [described, for example, in International Journal of Pharmaceutics 111, 223-233 (1994)], methotrexate, procarbazine, 6-mercaptopurine and mucophenolic acid.

### Metabolism:

Anorectic and weight reducing agents including dexfenfluramine, fenfluramine, diethylpropion, mazindol and phentermine.

Agents used in hypercalcaemia such as calcitriol, dihydrotachysterol and their active derivatives or analogs.

### Respiratory System:

Antitussives such as ethylmorphine, dextromethorphan and pholcodine.

Expectorants such as acetylcysteine, bromhexine, emetine, guaiphenesin, ipecacuanha ans saponins.

Decongestants such as phenylephrine, phenylpropanolamine and pseudoephedrine.

Bronchospasm relaxants such as ephedrine, fenoterol, orciprenaline, rimiterol, salbutamol, sodium cromoglycate, cromoglycic acid and its prodrugs [described, for example, in International Journal of Pharmaceutics 7, 63-75 (1980)], terbutaline, ipratropium bromide, salmeterol and theophylline and theophylline derivatives.

### Allergy and Immune System:

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine.

Local anaesthetics such as bupivacaine, amethocaine, lignocaine, cinchocaine, dibucaine, mepivacaine, prilocalne and etidocaine.

Stratum corneum lipids, such as ceramides, cholesterol and free fatty acids, for improved skin barrier repair [Man, et al. J. Invest. Dermatol., 106(5), 1096, 1996].

Neuromuscular blocking agents such as suxamethonium, alcuronium, pancuronium, atracurium, gallamine, tubocurarine and vecuronium.

Smoking cessation agents such as nicotine, bupropion and ibogaine.

Insecticides and other pesticides which are suitable for local or systemic application.

Dermatological agents, such as vitamins A and E, vitamin E acetate and vitamin E sorbate.

Allergens for desensitisation such as house dust mite allergen.

Nutritional agents, such as vitamins, essential amino acids and essential fats.

Keratolytics such as the alpha-hydroxy acids, glycollic acid and salicylic acid.

Psychicenergisers, such as 3-(2-aminopropyl)indole, 3-(2-aminobutyl)indole, and the like.

Anti-acne agents such as containing isotretinoin, tretinoin and benzoyl peroxide.

Anti-psoriasis agents such as containing etretinate, cyclosporin and calcipotriol.

Anti-itch agents such as capsaicin and its derivatives such as nonivamide [Tsai, et al. Drug. Dev. Ind. Pharm., 20(4), 719, 1994].

Anticholinergic agents, which are effective for the inhibition of axillary sweating and for the control of prickly heat. The antiperspirant activity of agents such as methatropine nitrate, propantheline bromide, scopolamine, methscopolamine bromide, and the new class of soft antiperspirants, quaternary acyloxymethyl ammonium salts [described, for example, by Bodor et al, J. Med. chem. 23, 474 (1980) and also in United Kingdom Specification No. 2010270, published Jun. 27, 1979].

Other physiologically active peptides and proteins, small to medium-sized peptides, e.g., vasopressin and human growth hormone.

Suitable dermal penetration enhancers in accordance with the present invention are preferably present in amounts ranging from about 0.1% to about 60% w/w, preferably between about 1% to about 40% w/w and more preferably between about 1% to about 20% w/w and include those dermal penetration enhancers that when used in effective amounts are non-irritating to the skin. Preferred dermal penetration enhancers include ester sunscreens, which are generally considered safe by the United States Food and Drug Administration (U.S. FDA).

The preferred ester dermal penetration enhancers include C₇₋₂₅ alkyl para-aminobenzoate, C₇₋₂₅ alkyl dimethyl-para-aminobenzoate, C₇₋₂₅ alkyl cinnamate, C₇₋₂₅ alkylmethoxycinnamate or C₇₋₂₅ alkyl salicylate; most preferably octyl dimethyl-para-aminobenzoate, octyl para-methoxycinnamate, octyl salicylate or isoamyl salicylate. Dermal penetration enhancers such as octyl dimethyl-para-aminobenzoate (Padimate O) and octyl salicylate have been extensively used over the last ten to twenty years as safe and effective sunscreens in concentrations up to about 8% v/v for Padimate O and about 5% v/v for octyl salicylate.

Preferred ether sunscreen dermal penetration enhancers of the present invention are of the structure illustrated in Formula 1 below.

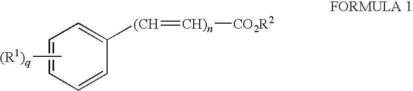

Formula 1 illustrates a structure wherein R¹ is selected from the group consisting of hydrogen, C₁₋₆ alkyl such as for example but not limited to methyl, propyl or butyl, C₁₋₆ alkoxy such as for example but not limited to methoxy, ethoxy or butoxy, halide such as for example but not limited to fluorine, chlorine or iodine, hydroxy and NR³R⁴; R² is a C₇₋₂₅ alkyl such as for example but not limited to heptyl or octyl; R³ and R⁴ may be the same or different selected from the group consisting of hydrogen and C₁₋₆ alkyl such as for example but not limited to methyl, propyl or butyl, or R³ and R⁴ together with the nitrogen atom to which they are bonded form a 5- or 6-membered heterocyclic ring; n is 0 or 1; and q is 1 or 2.

Other preferred dermal penetration enhancers include mono C₁₋₆ alkyl ethers of diethylene glycol such as for example but not limited to diethylene glycol monoethyl ether or diethylene glycol monomethyl ether. Mono C₁₋₆ alkyl ethers of diethylene glycol are preferred due to skin tolerance and acceptance by the U.S. FDA.

Other known dermal penetration enhancers could be used in the composition of the present invention. Such known dermal penetration enhancers include laurocapram (Azone®) and laurocapram derivatives, such as those 1-alkylazacycloheptan-2-ones disclosed in U.S. Pat. No. 5,196,410, and oleic acid and its ester derivatives, such as methyl, ethyl, propyl, isopropyl, butyl, vinyl and glycerylmonooleate, and sorbitan esters such as sorbitan monolaurate and sorbitan monooleate, and other fatty acid esters such as isopropyl laurate, isopropyl myristate, isopropyl palmitate, diisopropyl adipate, propylene glycol monolaurate and propylene glycol monooleate, and long chain alkyl esters of 2-pyrrolidone, particularly the 1-lauryl, 1-hexyl and 1-(2-ethylhexyl) esters of 2-pyrollidone, dodecyl (N,N-dimethylamino) acetate and dodecyl (N,N-dimethylamino) propionate and those dermal penetration enhancers disclosed in U.S. Pat. Nos. 5,082,866 and 2-n-nonyl-1-3-dioxolane disclosed in U.S. Pat. No. 4,861,764.

In certain aspects of the invention, the penetration enhancer may be selected from the groups including terpenes, terpenoids, essential oils, pyrrolidones, azones, fatty acids and esters, sulfoxides, amides, glycols and glycerides, amino acid derivatives, phospholipids, surfactants, cyclodextrin complexes, and other groups.

Penetration enhancers that are terpenes, terpenoids, or essential oils include l-menthol, eucalyptus oil, peppermint oil, turpentine oil, cineole, 1,8-cineole, eucalyptol, d-limonene, □-pinene, nerolidol, □-bisabolol, terpinol, 3-carene, terpinen-4-ol, carveol, carvone, pulgone, piperitone, menthone, cyclohexene oxide, limonene oxide, pinene oxide, cyclopentene oxide, ascaridole, and 7-oxabicyclo(2-2-1)heptane.

Penetration enhancers that are pyrrolidones and azones include N-methyl-2-pyrrolidone (NMP), 2-pyrrolidone, 1-propyl-3-dodecyl-2-pyrrolidone, 1-butyl-3-dodecyl-2-pyrrolidone, 1-ethyl-2-pyrrolidone, 1-hexyl-2-pyrrolidone, 1-butyl-2-pyrrolidone, 1-octyl-2-pyrrolidone, N-dodecyl-2-pyrrolidone, N-(2-hydroxyethyl)-2-pyrrolidone, 1-dodecylazacycloheptan-2-one (azone), 1-geranylazacycloheptone-2-one, 1-farnesylazacycloheptone-2-one, 1-geranylazacyclopentan-2,5-dione, 1-farnesylazacyclopentan-2-one, N-dodecyl-2-piperdinone, 2-(1-nonyl)-1,3-dioxolane (SEPA), cyclopentadecalactone (CPE-215), 1-[2-(decylthiolethyl)azacyclopentan-2-one (HPE-101), 4-decyloxazolid-2-one (Dermac SR-38).

The term azones, as used herein refers to 1-alkylazacycloheptan-2-one, wherein the alkyl group has from 8 to 16 carbon atoms. Penetration enhancers that are fatty acids and esters include, oleic acid, linoleic acid, capric acid, lauric acid, neodecanoic acid, myristic acid, fatty acid extract of cod liver oil, isopropylmyristate, valeric acid, heptanoic acid, pelargonic acid, isovaleric acid, neopentanoic acid, neoheptanoic acid, neononanoic acid, isostearic acid, myristoleic acid, palmitoleic acid, gondoic acid, erucic acid, a-linolenic acid, arachidonic acid, asclepic acid, petroselinic acid, elaidic acid and esters thereof. Preferred esters include alkyl esters, particularly those having from 6-24 carbon atoms, which may be unbranched or branched, saturated or unsaturated, and which may be cyclic or contain a cycloalkyl portion, and which may be unsubstituted or substituted with one or more substituents selected from lower alkoxy, hydroxyl, oxo, halo, and amino.

In certain preferred embodiments, the penetration enhancer is selected from a compound having the formula Iₐ or I_{b}:

R¹—C(═O)—O—H or R²—C(═O)—O—R³ Iₐ I_{b}

wherein:
R¹ is selected from a straight chain, branched, or cyclic-containing alkyl group or substituted alkyl group having 6 to 20 carbons, and a straight chain, branched, or cyclic-containing alkenyl group or substituted alkenyl group having 8 to 20 carbons; R² is selected from a straight chain, branched, or cyclic-containing alkyl group or substituted alkyl group having 6 to 20 carbons, and a straight chain, branched, or cyclic-containing alkenyl group or substituted alkenyl group having 8 to 20 carbons; and
R³ is selected from lower alkyl, lower alkenyl, a straight chain, branched, or cyclic-containing alkyl group or substituted alkyl group having 4 to 14 carbons, and a straight chain, branched, or cyclic-containing alkenyl group or substituted alkenyl group having 4 to 14 carbons; and
the substituted alkyls or substituted alkenyls have from 1 to 4 substituents selected from hydroxy, halo, oxo, alkoxy, and amino.

When the alkyl or alkenyl groups contain a cyclic portion, the cyclic portion may have from 3-7 carbon atoms in the ring. The cyclic portion may be saturated or may contain a double bond between adjacent carbons. Also, the cyclic portion may contain up to two hetero atoms (i.e., O, S, or N) in place of one of the 2-7 carbon atoms in the ring. The cyclic portion may be unsubstituted, or may be optionally substituted with 1 to 4 substituents selected from lower alkyl, lower alkoxy, hydroxyl, oxo, halo, and amino.

Alkyl and alkoxy groups referred to herein may be either straight chain or branched. The term “lower alkyl” refers to alkyl groups containing from 1 to 5 carbon atoms. The term lower alkoxy refers to the group —O-(lower alkyl). The term “halide” or “halo” means fluoride, chloride, bromide or iodide. The term “amino” refers to —NH₂, —NH(lower alkyl), or —N(lower alkyl)₂.

Penetration enhancers that are amides include dimethylacetamide, N,N-dimethyloctanamide, and N,N-dimethyldecanamide. Preferred amides have the formula II:

R⁴—C(═O)—N(R⁵)(R⁶) II

R⁴ is selected from a straight chain, branched, or cyclic-containing alkyl group or substituted alkyl group having 2 to 20 carbons, and a straight chain, branched, or cyclic-containing alkenyl group or substituted alkenyl group having 2 to 20 carbons;
R⁵ is selected from a straight chain, branched, or cyclic-containing alkyl group or substituted alkyl group having 1 to 16 carbons, and a straight chain, branched, or cyclic-containing alkenyl group or substituted alkenyl group having 1 to 16 carbons; and
R⁶ is selected from H, a straight chain, branched, or cyclic-containing alkyl group or substituted alkyl group having 1 to 14 carbons, and a straight chain, branched, or cyclic-containing alkenyl group or substituted alkenyl group having 2 to 14 carbons; and
the substituted alkyls or substituted alkenyls have from 1 to 4 substituents selected from hydroxy, halo, oxo, alkoxy, and amino.

Penetration enhancers that glycols and glycerides include: propylene glycol, glycerin tricaprylate (caprylic acid triglyceride), glyceryl monocaprylate, Sefsol 318 (medium-chain glyceride, monoglycerides, polyglycosylated glycerides, Transcutol, poyethylene glycol 400, and polycylcolized glyceride.

Penetration enhancers that are sulfoxides include: dimethyl sulfoxide, and decylmethyl sulfoxide. Preferred sulfoxides have the formula: (C₂-C₁₆ alkyl)-S(═O)—(C₁-C₁₆ alkyl), and particularly preferred sulfoxides have the formula (C₄-C₁₆ alkyl)-S(═O)—(C₁-C₃ alkyl).

Penetration enhancers that are amino acid derivatives include: N-dodecyl-1-amino acid methyl ester, n-pentyl-N-acetyl prolinate, octyl-6-aminohexanoate, decyl-6-aminohexanoate, dodecyl-N,N-dimethylamino isopropionate, and dodecyl-N,N-dimethylamino acetate.

Penetration enhancers that are phospholipids include: phosphatidyl glycerol derivatives, phosphatidyl choline derivates, and phosphatidyl ethanolamine derivatives.

Penetration enhancers that are surfactants include: bile salts, polysorbates, and sodium lauryl sulfate.

Penetration enhancers that are cyclodextrin complexes include: n-cyclodextrin and methyl-□-cyclodextrin.

Other preferred penetration enhancers include: alkyl-2-(N,N-disubstituted amino)-alkanoate ester (NexAct), N-acetylprolineesters, neohesperidinedihydrochalcone, fatty acid esters of lactic acid salts, polyethyleneglycol monoalkyl ethers, crotamiton, levulic acid, sterols and sterol esters, acyl lactylates, oleic acid dimers, neodecanoic acid, dioxolanes, polyoxyethylene cetyl ethers, methyl laurate, glycerol monolaurate, and esters and amides of clofibric acid.

In certain embodiments, particularly preferred penetration enhancers include: butylated hydroxyanisole, 2-phenoxyethanol, thymol, menthol, menthone, cineole, isopropyl myristate, glyceryl monolaurate, glyceryl monostearate, glyceryl monooleate, oleic acid, oleyl alcohol, methyl laurate, sorbitan monooleate, lauryl lactate, and lauryl alcohol.

In certain embodiments of the invention, preferred dermal penetration enhancers include fatty acids and fatty acid esters and derivatives thereof. In certain embodiments, the fatty acid portion of the fatty acid ester and the alcohol portion of the ester are selected from linear or branched alkyl groups.

While it is preferred that the active agent and penetration enhancer be delivered by simultaneous administration, the penetration enhancer may be applied before or after the application of the physiologically active agent, if desired. Likewise, if desired, diluents, carriers, surfactants, additives or the like may be added to the composition of the present invention.

The present invention, while providing a transdermal drug delivery composition, also provides a method for administering the composition to an animal, which for purposes of the present invention includes humans, comprising applying an effective dosage amount of the subject composition to a dermal or mucosal surface of an animal in need thereof. The present invention also provides a method for the treatment or prophylaxis of a disease or condition in an animal comprising administering to a dermal or mucosal surface of an animal in need thereof a therapeutically effective amount of the drug delivery composition of the present invention. Furthermore, the present invention provides a metered spray aerosol or pump drug delivery device of specified dimensions for controlled administration of a composition of the present invention to a dermal or mucosal surface of an animal. Preferably the animal is a human but the present invention also extends to the treatment of non-human animals.

Preferably, the non-occlusive, transdermal drug delivery composition of the present invention is not supersaturated with respect to the physiologically active agent or prodrug. As the non-volatile liquid of the non-occlusive drug delivery composition evaporates, the remaining composition components are rapidly absorbed into the dermal or mucosal surface. It is possible that as the non-volatile liquid evaporates, the dermal penetration enhancer becomes supersaturated with respect to the active agent. However, it is preferred that any supersaturation does not occur prior to absorption of the remaining components into the dermal or mucosal surface has occurred.

It is most desirable that, after administration of the transdermal drug delivery system of the present invention to a dermal or mucosal surface, the nonvolatile liquid component of the composition evaporates and the area of skin to which the drug delivery system was applied becomes visually dry. Said area of skin becomes visually dry within 10 minutes, preferably within 4 minutes, more preferably within 3 minutes and most preferably within 1 minute.

Suitable non-volatile liquids for use in compositions of the present invention include suitable carriers such as but not limited to deionized water, waters glycerides such as for example but not limited to medium-chain glycerides, monoglycerides and polyglycosylated glycerides, vegetable oils, mineral oils, silicone oils such as for example but not limited to dimethicone, animal oils such as for example but not limited to mink oil, benzoates such as for example but not limited to C₁₂₋₁₅ alkyl benzoates, ocytyl dodecyl benzoate and isostearyl benzoate and like carriers whereby deionized water is preferred. Certain non-volatile liquids suitable as carriers may also serve as penetration enhancers, such as for example but not limited to some glycerides.

In accordance with the present invention, the alcohol-free, transdermal drug delivery composition of the present invention is preferably packaged in an aerosol or pump device. An example of a transdermal drug delivery composition of the present invention is set forth below in Table 1.

TABLE I

| | | Weight % | | |
|---|---|---|---|---|
| Component | | Suitable | Preferred | Optimum |
| Active Agent: | Estradiol | 0.1-15% | 1-12% | 1-5% |
| Dermal Penetration | DGME | 0.1-60% | 1-40% | 1-20% |
| Enhancer: | | | | |
| Non-volatile Liquid: | DI Water | RW | RW | RW |
| Propellant: | DME | 35-50% | 35-40% | 35-38% |

DGME=Diethylene glycol monoethyl ether
RW=Substantially the remainder of the formulation is deionized water.
DME=Dimethyl ether

As set forth in Table 1 above, the propellant used in the aerosol device is dimethyl ether (DME). Small amounts of one or more other propellants could also be used, although less preferred. Such suitable propellants are those known to those skilled in the art including but not limited to dichlorodifluoromethane, trichlorofluoromethane, dichlorotetrafluoroethane, carbon dioxide or other suitable gases. At ambient temperature, i.e., 25° C. or 70° F., DME is 35% soluble in water, and water is 6% soluble in DME. In the example of Table 1, DME is present in excess of about 35%, preferably about 35-50%, and most preferably about 35-40%, by weight of the total fill of the device.

As set forth in Table 2 below, another example composition in accordance with the present invention is packaged in a non-aerosol metered “pump” device.

TABLE 2

| | | Weight % | | |
|---|---|---|---|---|
| Component | | Suitable | Preferred | Optimum |
| Active Agent: | Estradiol | 0.1-15% | 1-12% | 1-5% |
| Dermal Penetration | DGME | 0.1-60% | 1-40% | 1-20% |
| Enhancer: | | | | |
| Non-volatile Liquid: | Water | RW | RW | RW |

DGME=Diethylene glycol monoethyl ether
RW=Substantially the remainder of the formulation is water.

An aerosol transdermal drug delivery device of the present invention is prepared from 37.5 g. of the formulation provided in Table 2 by combining each of the components of the formulation and then pouring equal amounts of the formulation into four “2 oz” aluminum cans (75 ml). Then, a 1-1 gas buret is employed to add about 19 g. of dimethyl ether (DME) to each can, which had previously been fitted with a valve having the following dimensions:

| | |
|---|---|
| Stem orifice: | 0.011 inch; |
| Vapor tap: | 0.016 inch; and |
| Capillary tubing: | 0.040 inch internal diameter. |

Each container is then fitted with a valve actuator having a 0.018-inch exit orifice.

Non-aerosol transdermal drug delivery devices of the present invention are prepared from 37.5 g. of the formulation provided in Table 2 by combining each of the components of the formulation and then pouring equal amounted of the formulation into four “2 oz” glass or other suitable bottle (75 ml). The bottle is then fitted with a metered pump-type valve having the following dimensions:

| | |
|---|---|
| Stem orifice: | 0.011 inch; and |
| Capillary tubing: | 0.040 inch internal diameter. |

Each container is then fitted with a valve actuator having a 0.018 inch exit orifice.

A test spray may be made from each container, aerosol or non-aerosol, to ensure a suitable metered spray for purposes of the present invention. For example, one metered spray may equal one dose, or multiple sprays may equal one dose, depending on the physiologically active agent and the desired dosage amount. The spray rate is about 0.4 g/second. The particle size of the spray is about 50 microns. After the testing the spray, each container is capped with a plastic closure.

In the example aerosol transdermal drug delivery device described above, the excess DME floats on the composition and provides a source of propellant vapor to propel the composition through the vapor tap. Such provides a soft spray of the composition without valve clogging. Furthermore, the DME enables a uniform spray of composition and uniform, fine spray particles during evacuation of the entire fill contents of the can. DME also provides a relatively “dry” spray which decreases the drying time of the water-based composition spray to under four minutes, more preferably under three minutes, and most preferably under one minute, for most pharmaceutical applications depending on dosage amount required. The drying time is measured from the time the composition is sprayed onto the dermal or mucosal surface until the surface appears visually dry. Confirmation that the surface is dry may be determined by touching the dermal or mucosal surface with standard, non-waxed, laboratory tissue. If upon touching the laboratory tissue to the dosed surface does not transfer a visually detectable amount of composition to the tissue, it is confirmed dry.

The liquid phase of DME that floats on top of the composition provides a source of propellant vapor that only goes through the vapor tap since it has no access to the dip tube at the bottom of the can. A small amount of this liquid phase floating on top of the composition also can enter into the composition to maintain the maximum amount of DME in solution with the composition. When the pressure activated valve is activated, the composition comes out of the can via the dip tube. At the same time, some vapor from the propellant in solution with the composition will escape as vapor through the vapor tap. That “in solution” propellant thus is drawn from the can in two ways, i.e., (1) as a liquid through the dip tube, and (2) as a vapor through the vapor tap. Drawing the propellant from the can in these two ways changes the percentage of propellant in solution with the composition because a disproportionate amount of DME is removed in the form of vapor. Without a vapor tap in the device, the solution of 34% DME and 66% composition would come out through the dip tube leaving the same percentage in the can. However, the presence of the vapor tap changes this situation. With the vapor tap, DME vapor is also removed from the can through the vapor tap. The larger the vapor tap opening, the greater the change in the composition-DME solution. Furthermore, the smaller the dimension of the valve stem controlling the amount of liquid coming out of the can, the greater will be the change in the solution composition. In this invention, a predetermined ratio of vapor tap to valve stem dimensions assures that the spray administered to the dermal or mucosal surface is of a small particle size for fast drying, of a low flow rate for a soft spray, and of a uniform delivery for consistency in the spray throughout the entire contents of the can.

The predetermined delivery rate for the transdermal drug delivery composition of the present invention is about 0.20 g/second to about 0.25 g/second, at a particle size of about 50 microns±12 microns for a spray containing approximately 60% by weight of the composition and approximately 40% by weight of DME. The valve-actuator device preferably has a predetermined vapor tap dimension of about 0.013 inches to about 0.020 inches, a stem orifice of about 0.010 inches to about 0.020 inches and a nozzle orifice on the actuator of about 0.018 inches±about 10 percent.

The composition of the present invention is administered via a drug delivery device having a metered spray as described above. For administration of the subject composition to a dermal or mucosal surface, a spray nozzle portion of the drug delivery device is held perpendicular to the dermal or mucosal surface at a height of about 50 mm. A drug delivery device of the present invention may then be used as described below.

### Drug Delivery Device Method of Use:

1. Hold the drug delivery device upright in the palm of your preferred hand with your index finger resting gently on the actuator valve.

2. Hold the drug delivery device approximately 50 mm from the dermal or mucosal surface to be treated with the opening of the spray nozzle pointed at the surface.

3. Depress the actuator valve once and release the valve.

4. Repeat steps 1, 2 and 3 on a new area of surface until the correct number of doses have been administered.

5. Allow the applied composition to dry on the skin for approximately one to approximately four minutes.

During application of the composition to the dermal or mucosal surface, the soft spray deposits the active agent onto the skin such that when the spray hits the surface of the skin it does not undergo any appreciable bounce-back into the atmosphere. A defined dose of active agent and penetration enhancer is forced through a uniform spray nozzle at a constant pressure form a defined height to give a uniform dose per cm². A dose of the subject composition may be applied once daily, or multiple times per day depending upon the condition of the patient. The transdermal drug delivery composition of the present invention may be applied topically to any body part, such as the thigh, abdomen, shoulder, and upper arm. In one embodiment, a composition is applied to about a 3 inch by about 3 inch area of skin. The site of application may vary from dose to dose. For example, the composition may be applied to the thigh for the first dose, the upper arm for the second dose, and back to the thigh for the third dose. This may be advantageous in alleviating any sensitivity of the skin to repeated exposure to components of the composition.

Preferred dosage amount of composition are capable of delivering an effective amount of the selected active agent over a period of about 12 to about 24 hours. By an “effective” or “therapeutically effective” amount of an active agent is meant a nontoxic, but sufficient amount of the agent to provide the desired effect. However, it will be appreciated by those skilled in the art that the desired dose will depend on the specific active agent as well as on other factors; the minimum effective dose of each active agent is of course preferred to minimize the side effects associated treatment with the selected active agent(s). The formulation is preferably applied on a regularly timed basis so that administration of the active agents is substantially continuous.

### EXAMPLE 1

### 17-β-Oestradiol Metered-Dose Transdermal Aerosol

Concentration Active ingredient: 2% w/v 17-β-Oestradiol
Dermal penetration enhancer: 8% v/v Octyl dimethyl-para-aminobenzoate
Non-volatile liquid: 50% v/v deionized water
Propellant: 40% v/v Dimethyl ether to give a final formulation pressure of about 2.0 kp/cm² (30 psi).

One spray of 50 μl will apply 1 mg of 17-β-oestradiol over an area of approximately 10 cm² Three sprays will be administered to the forearm skin, applying a dose of 3 mg over approximately 30 cm².

### EXAMPLE 2

### Testosterone Metered-Dose Transdermal Aerosol

Concentration Active ingredient: 12% w/v Testosterone
Dermal penetration enhancer: 8% v/v Octyl dimethyl-para-aminobenzoate
Non-volatile liquid: 50% v/v Deionized water
Propellant: 35% v/v Dimethyl ether to give a final formulation pressure of approximately 2.4 kp/cm² (35 psi).

While the specification describes particular embodiments of the present invention, those of ordinary skill can devise variations of the present invention without departing from the inventive concept. Thus, the invention described and claimed herein is not to be limited in scope by the specific embodiments disclosed herein, since these embodiments are intended as illustrations of several aspects of the invention. Any equivalent embodiments are intended to be within the scope of the invention. Indeed, various modifications of the invention in addition to those shown and described herein will become apparent to those skilled in the art from the foregoing description. Such modifications are also intended to fall within the scope of the appended claims.

1. An alcohol-free composition comprising:
an effective amount of one or more physiologically active agents;
an effective amount of one or more dermal penetration enhancers selected from the group consisting of ethers of diethylene glycol and ester sunscreens; and one or more non-volatile liquids in an alcohol-free composition.

2. A drug delivery device comprising:
an effective amount of one or more physiologically active agents;
an effective amount of one or more dermal penetration enhancers; and
a non-volatile liquid in an alcohol-free composition contained within a valve-actuated metered spray drug delivery device.

3. The composition of claim 1 wherein said active agents are selected from the group consisting of androgens, anti-androgens, estrogens, anti-estrogens, progestogens, anti-progestogens, adrenergic agonists, analgesics, sedatives, amides, arylpiperazines, nerve agents, antineoplastics, anti-inflammatory agents, anticholinergics, anticonvulsants, antidepressants, antiepileptics, antihistaminics, antihypertensives, muscle relaxants, diuretics, bronchodilators, and glucocorticoids.

4. The composition of claim 1 wherein said active agents are selected from the group consisting of alimentary agents, cardiovascular agents, blood and haemopoietic agents, central nervous system agents, musculoskeletal agents, hormones, steroids, genitourinary agents, antimicrobials, metabolism agents, allergy and immune system agents and respiratory agents.

5. The composition of claim 1 wherein said active agent is 17-β-oestradiol.

6. The composition of claim 1 wherein said dermal penetration enhancers are selected from the group consisting of ether sunscreen penetration enhancers, mono C₁₋₆ alkyl ethers of diethylene glycol, laurocapram derivatives, laurocapram derivatives, oleic acid and its ester derivatives, sorbitan esters, fatty acid esters, esters of 2-pyrrolidone and 2-n-nonyl-1-3-dioxolane.

7. The composition of claim 1 wherein said dermal penetration enhancer is an ether of diethylene glycol.

8. The composition of claim 1 wherein said non-volatile liquids are selected from the group consisting of deionized water, water, glycerides, vegetable oils, mineral oils, silicone oils, animal oils and benzoates.

9. The composition of claim 1 wherein said non-volatile liquid is water or deionized water.

10. The composition of claim 1 wherein said active agent is present in an amount ranging from about 0.1 to about 15 weight percent.

11. The composition of claim 1 wherein said dermal permeation enhancer is present in an amount ranging from about 0.1 to about 60 weight percent.

12. The composition of claim 1 wherein said non-volatile liquid is present in an amount up to about 65 weight percent.

13. The composition of claim 1 wherein said composition additionally includes one or more propellants.

14. The composition of claim 1 wherein said composition additionally includes one or more propellants selected from the group consisting of dichlorodifluoromethane, trichlorofluoromethane, dichlorotetrafluoroethane, carbon dioxide and dimethyl ether.

15. The composition of claim 1 wherein said composition additionally includes a carbon dioxide or dimethyl ether propellant.

16. The device of claim 2 wherein said active agents are selected from the group consisting of androgens, anti-androgens, estrogens, anti-estrogens, progestogens, anti-progestogens, adrenergic agonists, analgesics, sedatives, amides, arylpiperazines, nerve agents, antineoplastics, anti-inflammatory agents, anticholinergics, anticonvulsants, antidepressants, antiepileptics, antihistaminics, antihypertensives, muscle relaxants, diuretics, bronchodilators, and glucocorticoids.

17. The device of claim 2 wherein said active agent is selected from the group consisting of alimentary agents, cardiovascular agents, blood and haemopoietic agents, central nervous system agents, musculoskeletal agents, hormones, steroids, genitourinary agents, antimicrobials, metabolism agents, allergy and immune system agents and respiratory agents.

18. The device of claim 2 wherein said active agent is 17-□-oestradiol.

19. The device of claim 2 wherein said dermal penetration enhancers are selected from the group consisting of ether sunscreen penetration enhancers, mono C1-6 alkyl ethers of diethylene glycol, laurocapram derivatives, laurocapram derivatives, oleic acid and its ester derivatives, sorbitan esters, fatty acid esters, esters of 2-pyrrolidone and 2-n-nonyl-1-3-dioxolane.

20. The device of claim 2 wherein said dermal penetration enhancer is an ether of diethylene glycol.

21. The device of claim 2 wherein said non-volatile liquids are selected from the group consisting of deionized water, water, glycerides, vegetable oils, mineral oils, silicone oils, animal oils and benzoates.

22. The device of claim 2 wherein said non-volatile liquid is water or deionized water.

23. The device of claim 2 wherein said active agent is present in an amount ranging from 0.1 to 15 weight percent.

24. The device of claim 2 wherein said dermal permeation enhancer is present in an amount ranging from 0.1 to 60 weight percent.

25. The device of claim 2 wherein said non-volatile liquid is present in an amount up to about 65 weight percent.

26. The device of claim 2 wherein said composition additionally includes one or more propellants.

27. The device of claim 2 wherein said composition additionally includes one or more propellants selected from the group consisting of dichlorodifluoromethane, trichlorofluoromethane, dichlorotetrafluoroethane, carbon dioxide and dimethyl ether.

28. The device of claim 2 wherein said composition additionally includes dimethyl ether or carbon dioxide as a propellant.

29. The device of claim 2 wherein said device includes a vapor tap dimensioned about 0.013 inches to about 0.020 inches.

30. The device of claim 2 wherein said device includes a stem orifice dimensioned about 0.010 inches to about 0.014 inches.

31. The device of claim 2 wherein said device includes a nozzle orifice dimensioned about 0.018+10 percent.

32. The device of claim 2 wherein said device upon actuation sprays said composition at a spray delivery rate of about 0.20 g/second to about 0.25 g/second.

33. The device of claim 2 wherein said device upon actuation sprays said composition with a particle size of approximately 50 microns.

34. A method of making an alcohol-free drug delivery composition comprising:
combining an effective amount of one or more physiologically
active agents; an effective amount of one or more dermal penetration enhancers selected from the group consisting of ethers of diethylene glycol and ester sunscreens; and one or more non-volatile liquids in an alcohol-free composition.

35. A method of administering an alcohol-free drug delivery composition comprising:
spraying an effective amount of one or more physiologically
active agents; an effective amount of one or more dermal penetration enhancers selected from the group consisting of ethers of diethylene glycol and ester sunscreens; and one or more non-volatile liquids in an alcohol-free composition onto a dermal or mucosal surface.

36. The method of claim 34 or 35 wherein said active agents are selected from the group consisting of androgens, anti-androgens, estrogens, anti-estrogens, progestogens, anti-progestogens, adrenergic agonists, analgesics, sedatives, amides, arylpiperazines, nerve agents, antineoplastics, anti-inflammatory agents, anticholinergics, anticonvulsants, antidepressants, antiepileptics, antihistaminics, antihypertensives, muscle relaxants, diuretics, bronchodilators, and glucocorticoids.

37. The method of claim 34 or 35 wherein said active agents are selected from the group consisting of alimentary agents, cardiovascular agents, blood and haemopoietic agents, central nervous system agents, musculoskeletal agents, hormones, steroids, genitourinary agents, antimicrobials, metabolism agents, allergy and immune system agents and respiratory agents.

38. The method of claim 34 or 35 wherein said active agent is 17-□-oestradiol.

39. The method of claim 34 or 35 wherein said dermal penetration enhancers are selected from the group consisting of ether sunscreen penetration enhancers, mono C1-6 alkyl ethers of diethylene glycol, laurocapram derivatives, laurocapram derivatives, oleic acid and its ester derivatives, sorbitan esters, fatty acid esters, esters of 2-pyrrolidone and 2-n-nonyl-1-3-dioxolane.

40. The method of claim 34 or 35 wherein said dermal penetration enhancer is an ether of diethylene glycol.

41. The method of claim 34 or 35 wherein said non-volatile liquids are selected from the group consisting of deionized water, water, glycerides, vegetable oils, mineral oils, silicone oils, animal oils and benzoates.

42. The method of claim 34 or 35 wherein said non-volatile liquid is water or deionized water.

43. The method of claim 34 or 35 wherein said active agent is present in an amount ranging from about 0.1 to about 15 weight percent.

44. The method of claim 34 or 35 wherein said dermal permeation enhancer is present in an amount ranging from about 0.1 to about 60 weight percent.

45. The method of claim 34 or 35 wherein said non-volatile liquid is present in an amount up to about 65 weight percent.

46. The method of claim 34 or 35 wherein said composition additionally includes one or more propellants.

47. The method of claim 34 or 35 wherein said composition additionally includes one or more propellants selected from the group consisting of dichlorodifluoromethane, tichlorofluoromethane, dichlorotetrafluoroethane, carbon dioxide and dimethyl ether.

48. The method of claim 34 or 35 wherein said composition additionally includes dimethyl ether or carbon dioxide as a propellant.

49. The method of claim 35 wherein said surface is dry in less than 10 minutes.

50. The method of claim 35 wherein said surface is dry in less than 4 minutes.

51. The method of claim 35 wherein said surface is dry in less than 3 minutes.

52. The method of claim 35 wherein said surface is dry in less than 1 minute.

53. A method for the treatment or prophylaxis of a disease or condition in an animal which comprises administering to a dermal or mucosal surface of said animal in need of such treatment a therapeutically effective amount of the composition of claim 1.

54. The method according to claim 53, wherein the disease or condition requires male hormone replacement therapy or requires female hormone replacement therapy.

55. The method according to claim 53, wherein the disease or condition is soft tissue injury, narcotic withdrawal, severe post-operative pain, motion sickness, oestrogen dependent breast cancer, prostatic enlargement and/or prostatic cancer, alopecia and acne, anxiety disorders, male impotence, Raynauds syndrome and varicose veins, sleep disorders, jetlag, herpes virus infections, deep vein thrombosis, migraine, high blood pressure, malaria, diagnosis of cystic fibrosis, asthma or nocturnal asthma.

56. A non-occlusive, transdermal drug delivery composition which comprises:
(i) a therapeutically effective amount of at least one physiologically active agent;
(ii) at least one dermal penetration enhancer, which is present in an amount of from 10 to 10,000 wt % based on the weight of the active agent;
(iii) at least one non-volatile liquid present in an amount to act as a vehicle for the active agent and penetration enhancer; wherein:
the dermal penetration enhancer (A) is adapted to transport the physiologically active agent across a dermal or mucosal surface of an animal, when the non-volatile liquid evaporates, to form a reservoir or depot of a mixture comprising the penetration enhancer and the physiologically active agent within said surface or membrane, and (B) is tolerated by the dermal or mucosal surface of the animal; and,
after administration of the composition to an area of the dermal or mucosal surface, the area becomes dry within 4 minutes of application.

57. A composition according to claim 56, wherein the dermal or mucosal surface becomes dry within one minute of application.

58. A composition according to claim 56, wherein said dermal permeation enhancer is an alkyl para-aminobenzoate, alkyl dimethyl-para-aminobenzoate, alkyl cinnamate, alkyl methoxycinnamate or alkyl salicylate.

59. A composition according to claim 56, wherein said dermal penetration enhancer is octyl dimethyl-para-aminobenzoate, octyl para-methoxycinnamate or octyl salicylate.

60. A composition according to claim 56, wherein the non-volatile liquid is deionized water.

61. A composition according to claim 56, wherein the physiologically active agent is a steroid, hormone derivative, non-steroidal anti-inflammatory drug, opioid analgesic, antinauseant, antioestrogen, aromatase inhibitor, 5-alpha reductase inhibitor, anxiolytic, prostaglandin, anti-viral drug, anti-migraine compound, antihypertensive agent, anti-malarial compound, bronchodilator anti-depressant, anti-alzheimer's agent, neuroleptic and antipsychotic agent, anti-parkinson's agent, antiandrogen or anorectic agent.

62. A composition according to claim 56, wherein the physiologically active agent is testosterone, oestradiol, ethinyloestradiol, progesterone, norethisterone acetate, ibuprofen, ketoprofen, flurbiprofen, naproxen, diclofenac, fentanyl, buprenorphine, scopolamine, prochlorperazine, metochlopramide, ondansetron, tamoxifen, epitiostanol, exemestane, 4-hydroxy-androstenedione and its derivatives, finasteride, turosteride, LY191704, MK-306, alprazolam, alprostadil, prostacylcin and its derivatives, melatonin, n-docosanol, tromantadine, lipophilic pro-drugs of acyclovir, low molecular weight heparin, enoxaparin, sumatriptan, amlodipine, nitrendipine, primaquine, minoxidil, minoxidil pro-drugs, pilocarpine, salbutamol, terbutaline, salmeterol, ibogaine, bupropian, rolipram, tacrine, fluphenazine, haloperidol, N-0923, cyproterone acetate or mazindol.

63. A composition according to claim 56, wherein the composition is applied to a dermal surface by a metered aerosol spray.

64. A composition according to claim 56, wherein the aerosol is a fixed or variable metered dose aerosol.

65. A composition according to claim 56, further comprising a pharmaceutical compounding agent, co-solvent, surfactant, emulsifier, antioxidant, preservative, stabiliser, diluent or a mixture of two or more of said components.
